(19) United States
(12) Patent Application Publication
(10) Pub. No.: US 2022/0370995 A1
(43) Pub. Date: Nov. 24, 2022

(54) MONONUCLEAR TRANSITION METAL COMPLEXES AND PHOTOCATALYSTS FOR CARBON DIOXIDE REDUCTION INCLUDING THE SAME

(71) Applicant: KYTECBIO CO., LTD, Seoul (KR)

(72) Inventor: Jinheung KIM, Seoul (KR)

(73) Assignee: KYTECBIO CO., LTD, Seoul (KR)

(21) Appl. No.: 17/623,610

(22) PCT Filed: Apr. 20, 2021
(86) PCT No.: PCT/KR2021/004945

§ 371 Date: Jul. 20, 2022

(30) Foreign Application Priority Data
Apr. 21, 2020 (KR) 10-2020-0048256

(51) Int. Cl. B01J 35/00; B01J 31/18; B01J 35/12

(52) U.S. Cl. CPC B01J 35/004; B01J 31/1825; B01J 35/12; B01J 2231/625; B01J 2531/0213; B01J 2531/847; B01J 2531/842; B01J 2531/845; B01J 2531/72

(57) ABSTRACT

The present application provides a mononuclear transition metal complex, a photocatalyst for carbon dioxide reduction including same, and a method for reducing carbon dioxide to formic acid, the method comprising using the photocatalyst for carbon dioxide reduction.

### TECHNICAL FIELD

The present disclosure provides a mononuclear transition metal complex, a photocatalyst for carbon dioxide reduction including same, and a method for reducing carbon dioxide to formic acid, the method including using the photocatalyst for carbon dioxide reduction.

### BACKGROUND ART

Due to the high demand for the development of renewable and sustainable energy sources, the photochemical and electrochemical conversion of carbon dioxide into useful fuels has been studied for a longtime. Recently, since carbon dioxide is a real cause of global climate change and a potential carbon component of fine chemicals, intensive research has been conducted to study efficient catalyst systems for carbon dioxide conversion. For example, low-pressure photocatalytic reduction of carbon dioxide to formic acid and derivatives thereof is a very desirable conversion because these systems have been a focal point of carbon capture and photo-energy storage strategies and formic acid is used throughout the chemical industry as a reducing agent, acid and carbon source.

The scientific challenges facing the photocatalytic reduction of carbon dioxide include increasing efficiency and minimizing competitive reduction of water to H₂, which is a preferred process for the reaction rate kinetically over carbon dioxide reduction. In the past decades, molecular metal catalysts for the photochemical reduction of carbon dioxide have rarely been reported compared to the molecular metal catalysts for electrochemical carbon dioxide reduction. Among the few reported photochemical catalysts, most are polypyridine-based complexes of second- and third-row transition metals such as Ru, Re and Ir; most of these catalysts exhibited low turnover number (TON) and/or low selectivity. Meanwhile, a demand for the development of photo-driven carbon dioxide reduction catalysts based on earth-abundant transition metals for practical photochemical applications has increased. Molecular catalysts based on Fe, Co and Ni complexes containing tetradentate ligands have been developed for the visible light-driven reduction of carbon dioxide to CO. It was recently reported that a Ni carbine-isoquinoline complex exhibits a high TON of 98,000 for CO. In terms of formic acid photogeneration using earth-abundant metal complexes, it has been reported that the Mn-based catalyst, Mn (bpy) (CO)₃Br exhibited a TON of 157 for 12 hours, and a Fe catalyst with high catalyst selectivity containing a pentadentate ligand exhibited a TON of 5 for 20 hours. However, further embodiments of earth-abundant metal catalysts for substantial conversion of light-driven carbon dioxide into formic acid with high selectivity and high TON are desired.

As observed for [NiFe]-hydrogenases, Ni (II) complexes with N/S ligation have been closely studied due to their bioimpact properties. A series of mononuclear Ni-thiolate complexes such as Ni (bpy) (pyS)₂ and Ni (pyS)₃⁻ (pyS=pyridine-2-thiolate) have been investigated for photocatalytic and electrocatalytic H₂ production; some of these catalysts exhibited TONs greater than 5,000 for H₂ production. However, molecular Ni thiolate complexes as in the present disclosure have not been carefully investigated for photocatalytic carbon dioxide reduction.

### RELATED ART DOCUMENT

Korean Patent Laid-Open Publication No. 10-2017-0072531

### DISCLOSURE

### Technical Problem

The present disclosure provides a mononuclear transition metal complex, a photocatalyst for carbon dioxide reduction including same, and a method for reducing carbon dioxide to formic acid, the method comprising using the photocatalyst for carbon dioxide reduction.

However, an object to be solved by the present disclosure is not limited to those mentioned above, and the other objects not mentioned will be clearly understood by those skilled in the art from the following description.

### Technical Solution

A first aspect of the present disclosure is to provide a mononuclear transition metal complex represented by the following Formula 1:

L¹-M-(L²)₂ [Formula 1]

wherein

M is a mononuclear transition metal of Ni, Fe, Mn, or Co,

L¹ is

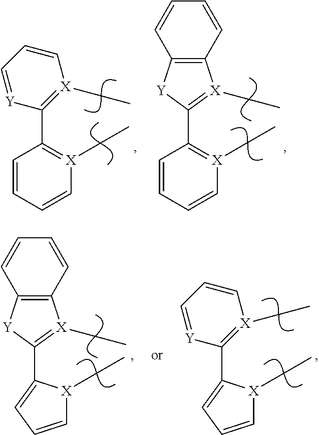

L² is

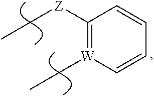

in L¹,

X is N or P,

Y is —CH, —N—, —NH, —S—, or —O—;

in L²,

Z is —O—, —S—, or —NH,

W is —P— or —N—;

the aryl group and/or heteroaryl group included in L¹ or L² is substituted or unsubstituted, and when the aryl group and/or the heteroaryl group is substituted, the substituent is one or more selected from a linear or branched C₁-C₆ alkyl group, a C₃-C₆ cycloalkyl group, a C₂⁻C₆ heterocycloalkyl group, a linear or branched C₁-C₆ alkoxy group, a halogen group, an amine group, or a linear or branched C₁-C₆ alkylamine group, and

the broken line means that the ligand is coordinated to the mononuclear transition metal.

A second aspect of the present disclosure is to provide a photocatalyst for carbon dioxide reduction, including a mononuclear transition metal complex represented by the following Formula 1:

L¹-M-(L²)₂ [Formula 1]

wherein

M is a mononuclear transition metal of Ni, Fe, Mn, or Co,

L¹ is

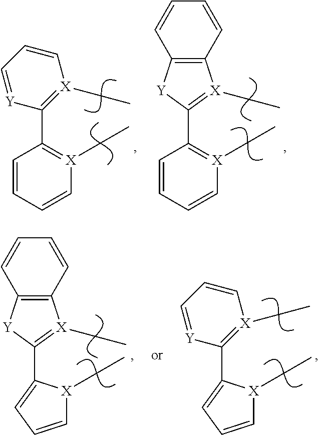

L² is

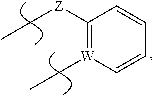

in L¹,

X is N or P,

Y is —CH, —N—, —NH, —S—, or —O—;

in L²,

Z is —O—, —S—, or —NH,

W is —P— or —N—;

the aryl group and/or heteroaryl group included in L¹ or L² is substituted or unsubstituted, and when the aryl group and/or the heteroaryl group is substituted, the substituent is one or more selected from a linear or branched C₁-C₆ alkyl group, a C₃-C₆ cycloalkyl group, a C₂⁻C₆ heterocycloalkyl group, a linear or branched C₁-C₆ alkoxy group, a halogen group, an amine group, or a linear or branched C₁-C₆ alkylamine group, and

the broken line means that the ligand is coordinated to the mononuclear transition metal.

A third aspect of the present disclosure is to provide a method for reducing carbon dioxide to formic acid, the method comprising using the photocatalyst for carbon dioxide reduction according to the second aspect.

### Advantageous Effects

According to the embodiments of the present disclosure, a photocatalyst for carbon dioxide reduction including a novel mononuclear transition metal complex optionally provides formic acid with high efficiency [about 14,000 turnover number].

According to the embodiments of the present disclosure, the photocatalyst for carbon dioxide reduction including a novel mononuclear transition metal complex provides a high catalytic selectivity of about 90% or more, about 95% or more, about 97% or more, about 98% or more, or about 99% or more.

According to the embodiments of the present disclosure, a photocatalyst for carbon dioxide reduction including a novel mononuclear transition metal complex may completely inhibit undesirable proton reduction paths in a photocatalytic reaction with mononuclear transition metals under carbon dioxide. Specifically, it is possible to inhibit the competitive reduction reaction of water to H₂ in the presence of carbon dioxide.

According to the embodiments of the present disclosure, a photocatalyst for carbon dioxide reduction including a novel mononuclear transition metal complex may be used to design a fuel production process through sunlight for artificial photosynthesis.

### DESCRIPTION OF DRAWINGS

FIG. 1a illustrates hydrogen photogeneration using Complex 1, EY (2.0 mM) and TEOA (400 mM) in EtOH:H₂O (1:1, pH=10.7) under argon (●) and carbon dioxide (▪), respectively, according to an embodiment of the present disclosure, and FIG. 1b illustrates hydrogen photogeneration using Complex 2 (4.0 μM), EY (2.0 mM) and TEOA (400 mM) in EtOH:H₂O (1:1, pH=10.7) under argon (●) and carbon dioxide (▪), respectively, according to an embodiment of the present disclosure.

FIG. 2 illustrates photocatalytic carbon dioxide conversion using Complex 1 (4.0 μM), EY (2.0 mM) and TEOA (400 mM) under different pH conditions in EtOH:H₂O (1:1) under carbon dioxide according to an embodiment of the present disclosure.

FIG. 3 illustrates hydrogen photogeneration using Complex 1 (4.0 μM), EY (2.0 mM) and TEOA (400 mM) in EtOH:H₂O (1:1) at pH 7.0 under argon (▪) and carbon dioxide (●), respectively, using a 420 nm cut-off filter according to an embodiment of the present disclosure.

FIG. 4a illustrates kinetic isotopic effects on formic acid photoproduction by Complex 1, EY (2.0 mM) and TEOA (400 mM) in EtOH:H₂O and EtOH/D₂O (1:1, pH=10.7), respectively, under carbon dioxide according to an embodiment of the present disclosure, and FIG. 4b illustrates kinetic isotopic effects on H₂ photogeneration by Complex 1, EY (2.0 mM) and TEOA (400 mM) in EtOH:H₂O and EtOH/D₂O (1:1, pH=10.7), respectively, under Ar according to an embodiment of the present disclosure.

FIG. 5a is a graph illustrating substantial kinetic isotopic effects on formic acid photogeneration by Complex 2, EY (2.0 mM) and TEOA (400 mM) in EtOH:H₂O/D₂O (1:1, pH=10.7) under carbon dioxide according to an embodiment of the present disclosure, and FIG. 5b is a graph illustrating kinetic isotopic effects on hydrogen photogeneration by Complex 2, EY (2.0 mM) and TEOA (400 mM) in EtOH:H₂O/D₂O (1:1, pH=10.7) under Ar according to an embodiment of the present disclosure.

FIG. 6a illustrates photocatalytic reduction of carbon dioxide to formic acid using Complex 1 (▪) and Complex 2 (●) (4.0 μM) in the presence of EY (2.0 mM) and TEOA (400 mM) in EtOH/H₂O (1:1, pH=10.7) at room temperature according to an embodiment of the present disclosure, and FIG. 6b is a cyclic voltammetry curve of Complex 1 under Ar (dotted line) and carbon dioxide (solid line) in 0.1 M KNO₃ (aq) (GC electrode, 100 mVs⁻¹) according to an embodiment of the present disclosure.

FIG. 7 illustrates a cyclic voltammetry curve (GC electrode, 100 mV/s) of Complex 2 in 0.1 M KNO₃ (aq) under argon (dotted line) and CO₂ (solid line) according to an embodiment of the present disclosure.

FIG. 8 illustrates an ORTEP photograph of Composite 2 according to an embodiment of the present disclosure.

FIG. 9 illustrates absorption spectra of 0.1 mM Complex 1 (solid line) and Complex 2 (dotted line) in H₂O/EtOH according to an embodiment of the present disclosure.

### BEST MODE FOR INVENTION

Hereinafter, the embodiments and examples of the present disclosure will be described in detail with reference to the accompanying drawings so as to be easily carried out by those of ordinary skill in the art to which the present disclosure pertains. However, the present disclosure may be implemented in various different forms and is not limited to the embodiment and examples described herein. In addition, in the drawings, portions unrelated to the description will be omitted to obviously describe the present disclosure, and similar portions will be denoted by similar reference numerals throughout the specification.

Throughout the present specification, when any one part is referred to as being “connected to” another part, it means that any one part and another part are “directly connected to” each other or are “electrically connected to” each other with the other part interposed therebetween.

Throughout the present specification, when any member is referred to as being positioned “on” another member, it includes not only a case in which any member and another member are in contact with each other, but also a case in which the other member is interposed between any member and another member.

Throughout the present specification, “including” any component will be understood to imply the inclusion of other components rather than the exclusion of other components, unless explicitly described to the contrary.

As used in the present specification, the terms “about,” “substantially,” etc., denoting degree, are used in a sense at or close to a numerical value when manufacturing and material tolerances inherent in the stated meaning are presented, and are used to prevent unfair use by an unscrupulous infringer of the disclosure in which exact or absolute numerical values are mentioned to aid understanding of the present disclosure.

As used throughout the present specification, the term “a step ˜” or “a step of ˜,” denoting degree, does not mean “a step for ˜”.

Throughout the present specification, the term “alkyl” or “alkyl group” refers to a linear or branched alkyl group having 1 to 12 carbon atoms, 1 to 10 carbon atoms, 1 to 8 carbon atoms, or 1 to 5 carbon atoms, and all possible isomers thereof.

For example, the alkyl or alkyl group may include, but are not limited to, a methyl group (Me), an ethyl group (Et), an n-propyl group (ⁿPr), an iso-propyl group (ⁱPr), an n-butyl group (ⁿBu), iso-butyl group (ⁱBu), a tert-butyl group (^{t}Bu), sec-butyl group (^{sec}Bu), an n-pentyl group (ⁿPe), an iso-pentyl group (^{iso}Pe), a sec-pentyl group (^{sec}Pe), a tert-pentyl group (^{t}Pe), a neo-pentyl group (^{neo}Pe)a 3-pentyl group, an n-hexyl group, an iso-hexyl group, a heptyl group, a 4,4-dimethylpentyl group, an octyl group, a 2,2,4-trimethylpentyl group, a nonyl group, a decyl group, an undecyl group, a dodecyl group, and isomers thereof, etc.

Throughout the present specification, the term “aryl” refers to a polyunsaturated, aromatic, hydrocarbon substituent which may be a single ring or multiple rings (1 to 3 rings) fused or covalently bonded together, unless otherwise indicated.

Specific examples of the aryl may include, but are not limited to, phenyl, naphthyl, biphenyl, anthryl, indenyl, fluorenyl, phenanthryl, triphenylenyl, pyrenyl, peryleneyl, chrysenyl, naphthacenyl, triazinyl, etc.

Throughout the present specification, the term “heteroaryl” refers to an aryl group (or ring) including 1 to 4 selected from heteroatoms such as N, O, P, and S (in each separate ring in the case of multiple rings) wherein the nitrogen and sulfur atoms are optionally oxidized and the nitrogen atom(s) are optionally quaternized. Specific examples of the heteroaryl may include, but are not limited to, monocyclic heteroaryl, such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, polycyclic heteroaryl such as benzofuranyl, benzothiophenyl, isobenzofuranyl, benzoimidazolyl, benzothiazolyl, benzoisothiazolyl, benzoisoxazolyl, benzooxazolyl, isoindolyl, indolyl, indazolyl, benzothiadiazolyl, quinolyl, isoquinolyl, cinnolinyl, quinazolinyl, quinoxalinyl, carbazolyl, phenanthridinyl, and benzodioxolyl, and corresponding N-oxides thereof (e.g., pyridyl N-oxide, quinolyl N-oxide), quaternary salts thereof, etc.

Throughout the present specification, the term “combination(s) thereof” included in the expression of a Markush-type refers to one or more mixtures or combinations selected from the group consisting of the components described in the expression of the Markush-type, which include one or more selected from the group consisting of the above components.

Throughout the present specification, the description of “A and/or B” refers to “A or B, or A and B.”

Hereinafter, embodiments and examples of the present disclosure will be described in detail with reference to the accompanying drawings. However, the present disclosure is not limited to these embodiments, examples and drawings.

A first aspect of the present disclosure is to provide a mononuclear transition metal complex represented by the following Formula 1:

L¹-M-(L²)₂ [Formula 1]

wherein

M is a mononuclear transition metal of Ni, Fe, Mn, or Co,

L¹ is

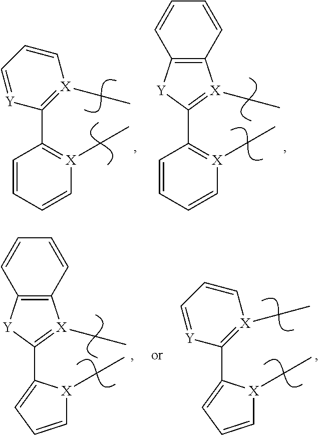

L² is

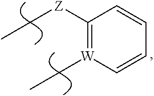

in L¹,

X is N or P,

Y is —CH, —N—, —NH, —S—, or —O—;

in L²,

Z is —O—, —S—, or —NH,

W is —P— or —N—;

the aryl group and/or heteroaryl group included in L¹ or L² is substituted or unsubstituted, and when the aryl group and/or the heteroaryl group is substituted, the substituent is one or more selected from a linear or branched C₁-C₆ alkyl group, a C₃-C₆ cycloalkyl group, a C₂-C₆ heterocycloalkyl group, a linear or branched C₁-C₆ alkoxy group, a halogen group, an amine group, or a linear or branched C₁-C₆ alkylamine group, and

the broken line means that the ligand is coordinated to the mononuclear transition metal.

In an embodiment of the present disclosure, the C₁-C₆ alkyl group may include, but are not limited to, a methyl group (Me), an ethyl group (Et), an n-propyl group (ⁿPr), an iso-propyl group (ⁱPr), a 1,1-dimethyl-n-propyl group, a 1,2-dimethyl-n-propyl group, a 2,2-dimethyl-n-propyl group, an 1-ethyl-n-propyl group, a 1,1,2-trimethyl-n-propyl group, a 1,2,2-trimethyl-n-propyl group, an 1-ethyl-1-methyl-n-propyl group, an 1-ethyl-2-methyl-n-propyl group, an n-butyl group (ⁿBu), an iso-butyl group (ⁱBu), a tert-butyl group (tert-Bu, ^{t}Bu), a sec-butyl group (sec-Bu, ^{sec}Bu), a 1-methyl-n-butyl group, a 2-methyl-n-butyl group, a 3-methyl-n-butyl group, a 1,1-dimethyl-n-butyl group, a 1,2-dimethyl-n-butyl group, a 1,3-dimethyl-n-butyl group, a 2,2-dimethyl-n-butyl group, a 2,3-dimethyl-n-butyl group, a 3,3-dimethyl-n-butyl group, an 1-ethyl-n-butyl group, an 2-ethyl-n-butyl group, an n-pentyl group (ⁿPe), an iso-pentyl group (^{iso}Pe), a sec-pentyl group (^{sec}Pe), a tert-pentyl group (^{t}Pe), a neo-pentyl group (^{neo}Pe), a 3-pentyl group, a 1-methyl-n-pentyl group, a 2-methyl-n-pentyl group, a 3-methyl-n-pentyl group, a 4-methyl-n-pentyl group, an n-hexyl group, an iso-hexyl group, or isomers thereof.

In an embodiment of the present disclosure, the C₃-C₆ cycloalkyl group may include, but are not limited to, a cyclopropyl group, 1-n-propyl-cyclopropyl group, an 2-n-propyl-cyclopropyl group, an 1-iso-propyl-cyclopropyl group, an 2-iso-propyl-cyclopropyl group, a 1,2,2-trimethyl-cyclopropyl group, a 1,2,3-trimethyl-cyclopropyl group, a 2,2,3-trimethyl-cyclopropyl group, an 1-ethyl-2-methyl-cyclopropyl group, an 2-ethyl-l-methyl-cyclopropyl group, an 2-ethyl-2-methyl-cyclopropyl cyclobutyl group, an 1-ethyl-cyclobutyl group, an 2-ethyl-cyclobutyl group, an 3-ethyl-cyclobutyl group, a 1,2-dimethyl-cyclobutyl group, a 1,3-dimethyl-cyclobutyl group, a 2,2-dimethyl-cyclobutyl group, a 2,3-dimethyl-cyclobutyl group, a 2,4-dimethyl-cyclobutyl group, a 3,3-dimethyl-cyclobutyl group, a cyclopentyl group, a 1-methyl-cyclopentyl group, a 2-methyl-cyclopentyl group, a 3-methyl-cyclopentyl group, a 1-methyl-cyclopentyl group, a 2-methyl-cyclopentyl group, a 3-methyl-cyclopentyl group, or a cyclohexyl group.

In an embodiment of the present disclosure, the halogen group may include, but are not limited to, fluorine (F), chlorine (Cl), bromine (Br) or iodine (I).

In an embodiment of the present disclosure, L¹ may be

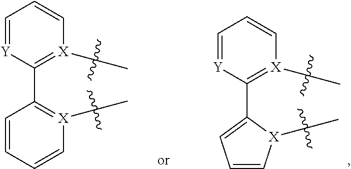

and Y may be —CH or —N—, but the present disclosure is not limited thereto.

In an embodiment of the present disclosure, L¹ may be

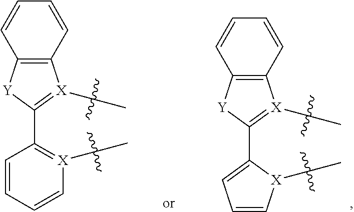

and Y may be NH, —S—, or —O—, but the present disclosure is not limited thereto.

In an embodiment of the present disclosure, the mononuclear transition metal complex represented by Formula 1 maybe (2-(2-pyridyl)benzothiazole)Ni(pyridine-2-thiolate)₂, (2-(2-pyridyl)benzimidazole)Ni(pyridine-2-thiolate)₂, (2-(2-pyridyl)benzothiazole)Co(pyridine-2-thiolate)₂, (2-(2-pyridyl))benzimidazole)Co(pyridine-2-thiolate)₂, (2-(2-pyridyl)benzothiazole)Mn(pyridine-2-thiolate)₂, (2-(2-pyridyl)benzimidazole)Mn(pyridine-2-thiolate)₂, (2-(2-pyridyl)benzothiazole)Fe(pyridine-2-thiolate)₂, or (2-(2-pyridyl)benzimidazole)Fe(pyridine-2-thiolate)₂, but the present disclosure is not limited thereto. Specifically, in an embodiment of the present disclosure, the mononuclear transition metal complex represented by Formula 1 may be (2-(2-pyridyl)benzothiazole)Ni(pyridine-2-thiolate)₂ or (2-(2-pyridyl)benzimidazole)Ni(pyridine-2-thiolate)₂.

In an embodiment of the present application, the transition metal of the mononuclear transition metal complex may be 6-coordinated in a distorted octahedral structure, but the present disclosure is not limited thereto.

A second aspect of the present disclosure is to provide a photocatalyst for carbon dioxide reduction, including a mononuclear transition metal complex represented by the following Formula 1:

L¹-M-(L²)₂ [Formula 1]

wherein

M is a mononuclear transition metal of Ni, Fe, Mn, or Co,

L¹ is

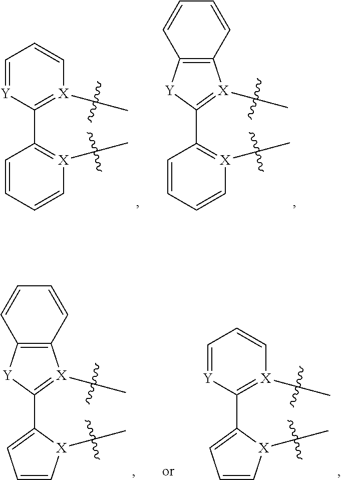

L² is

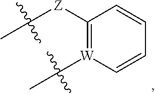

in L¹,

X is N or P,

Y is —CH, —N—, —NH, —S—, or —O—;

in L²,

Z is —O—, —S—, or —NH,

W is —P— or —N—;

the aryl group and/or heteroaryl group included in L¹ or L² is substituted or unsubstituted, and when the aryl group and/or the heteroaryl group is substituted, the substituent is one or more selected from a linear or branched C₁-C₆ alkyl group, a C₃-C₆ cycloalkyl group, a C₂⁻C₆ heterocycloalkyl group, a linear or branched C₁-C₆ alkoxy group, a halogen group, an amine group, or a linear or branched C₁-C₆ alkylamine group, and

the broken line means that the ligand is coordinated to the mononuclear transition metal.

Accordingly, detailed descriptions of parts overlapping with the first aspect of the present disclosure are omitted, but the contents described with respect to the first aspect of the present disclosure may be equally applied even if the description thereof is omitted in the second aspect of the present disclosure.

In an embodiment of the present disclosure, the C₁-C₆ alkyl group may include, but are not limited to, a methyl group (Me), an ethyl group (Et), an n-propyl group (ⁿPr), an iso-propyl group (ⁱPr), a 1,1-dimethyl-n-propyl group, a 1,2-dimethyl-n-propyl group, a 2,2-dimethyl-n-propyl group, an 1-ethyl-n-propyl group, a 1,1,2-trimethyl-n-propyl group, a 1,2,2-trimethyl-n-propyl group, an 1-ethyl-1-methyl-n-propyl group, an 1-ethyl-2-methyl-n-propyl group, an n-butyl group (ⁿBu), an iso-butyl group (ⁱBu), a tert-butyl group (tert-Bu, ^{t}Bu), a sec-butyl group (sec-Bu, ^{sec}Bu) a 1-methyl-n-butyl group, a 2-methyl-n-butyl group, a 3-methyl-n-butyl group, a 1,1-dimethyl-n-butyl group, a 1,2-dimethyl-n-butyl group, a 1,3-dimethyl-n-butyl group, a 2,2-dimethyl-n-butyl group, a 2,3-dimethyl-n-butyl group, a 3,3-dimethyl-n-butyl group, an 1-ethyl-n-butyl group, an 2-ethyl-n-butyl group, an n-pentyl group (ⁿPe), an iso-pentyl group (^{iso}Pe), a sec-pentyl group (^{sec}Pe), a tert-pentyl group (^{t}Pe), a neo-pentyl group (^{neo}Pe ), a 3-pentyl group, a 1-methyl-n-pentyl group, a 2-methyl-n-pentyl group, a 3-methyl-n-pentyl group, a 4-methyl-n-pentyl group, an n-hexyl group, an iso-hexyl group, or isomers thereof.

In an embodiment of the present disclosure, the C₃-C₆ cycloalkyl group may include, but are not limited to, a cyclopropyl group, 1-n-propyl-cyclopropyl group, an 2-n-propyl-cyclopropyl group, an 1-iso-propyl-cyclopropyl group, an 2-iso-propyl-cyclopropyl group, a 1,2,2-trimethyl-cyclopropyl group, a 1,2,3-trimethyl-cyclopropyl group, a 2,2,3-trimethyl-cyclopropyl group, an 1-ethyl-2-methyl-cyclopropyl group, an 2-ethyl-1-methyl-cyclopropyl group, an 2-ethyl-2-methyl-cyclopropyl cyclobutyl group, an 1-ethyl-cyclobutyl group, an 2-ethyl-cyclobutyl group, an 3-ethyl-cyclobutyl group, a 1,2-dimethyl-cyclobutyl group, a 1,3-dimethyl-cyclobutyl group, a 2,2-dimethyl-cyclobutyl group, a 2,3-dimethyl-cyclobutyl group, a 2,4-dimethyl-cyclobutyl group, a 3,3-dimethyl-cyclobutyl group, a cyclopentyl group, a 1-methyl-cyclopentyl group, a 2-methyl-cyclopentyl group, a 3-methyl-cyclopentyl group, a 1-methyl-cyclopentyl group, a 2-methyl-cyclopentyl group, a 3-methyl-cyclopentyl group, or a cyclohexyl group.

In an embodiment of the present disclosure, the halogen group may be fluorine (F), chlorine (Cl), bromine (Br) or iodine (I), but the present disclosure is not limited to thereto.

In an embodiment of the present disclosure, the photocatalyst for carbon dioxide reduction may reduce carbon dioxide to formic acid, but the present disclosure is not limited thereto.

In an embodiment of the present disclosure, the photocatalyst for carbon dioxide reduction further include a cocatalyst, wherein the cocatalyst may include, but is not limited to, one or more selected from eosin Y, Ru(bpy)₃, C₃N₄, CdS, CdSe, and triethanolamine. In an embodiment of the present disclosure, the photocatalyst for carbon dioxide reduction further include a cocatalyst, wherein the cocatalyst may include, but is not limited to, one or more selected from eosin Y and/or triethanolamine. Specifically, in an embodiment of the present disclosure, the photocatalyst may include eosin Y (EY) and triethanolamine (TEOA) as cocatalysts. Here, the eosin Y, Ru (bpy)₃, C₃N₄, CdS, or CdSe may function as a photosensitizer, and the triethanolamine may function as a sacrificial electron donor.

In an embodiment of the present disclosure, L¹ may be

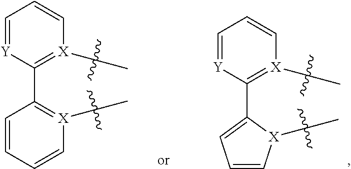

and Y may be —CH or —N—, but the present disclosure is not limited thereto.

In an embodiment of the present disclosure, L¹ may be

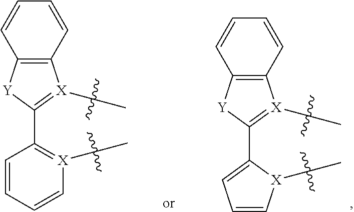

and Y may be NH, —S—, or —O—, but the present disclosure is not limited thereto.

In an embodiment of the present disclosure, the mononuclear transition metal complex may be one or more selected from (2-(2-pyridyl)benzothiazole)Ni(pyridine-2-thiolate)₂, (2-(2-pyridyl)benzimidazole)Ni(pyridine-2-thiolate)₂, (2-(2-pyridyl)benzothiazole)Co(pyridine-2-thiolate)₂, (2-(2-pyridyl))benzimidazole)Co(pyridine-2-thiolate)₂, (2-(2-pyridyl)benzothiazole)Mn(pyridine-2-thiolate)₂, (2-(2-pyridyl)benzimidazole)Mn(pyridine-2-thiolate)₂, (2-(2-pyridyl)benzothiazole)Fe(pyridine-2-thiolate)₂, and (2-(2-pyridyl)benzimidazole)Fe(pyridine-2-thiolate)₂, but the present disclosure is not limited thereto. Specifically, in an embodiment of the present disclosure, the mononuclear transition metal complex may be (2-(2-pyridyl)benzothiazole)Ni(pyridine-2-thiolate)₂ or (2-(2-pyridyl)benzimidazole)Ni(pyridine-2-thiolate)₂.

According to the embodiments of the present disclosure, a photocatalyst for carbon dioxide reduction including a novel mononuclear transition metal complex optionally provides formic acid with high efficiency [about 14,000 turnover number].

According to the embodiments of the present disclosure, the photocatalyst for carbon dioxide reduction including a novel mononuclear transition metal complex provides a high catalytic selectivity of about 90% or more, about 95% or more, about 97% or more, about 98% or more, or about 99% or more.

According to the embodiments of the present disclosure, a photocatalyst for carbon dioxide reduction including a novel mononuclear transition metal complex may completely inhibit undesirable proton reduction paths in a photocatalytic reaction with mononuclear transition metals under carbon dioxide. Specifically, it is possible to inhibit the competitive reduction reaction of water to H₂ in the presence of carbon dioxide.

According to the embodiments of the present disclosure, a photocatalyst for carbon dioxide reduction including a novel mononuclear transition metal complex may be used to design a fuel production process through sunlight for artificial photosynthesis.

A third aspect of the present disclosure is to provide a method for reducing carbon dioxide to formic acid, the method including using the photocatalyst for carbon dioxide reduction according to the second aspect.

Accordingly, detailed descriptions of parts overlapping with the first and second aspects of the present disclosure are omitted, but the contents described with respect to the first and second aspects of the present disclosure may be equally applied even if the description is omitted in the third aspect of the present disclosure.

In an embodiment of the present disclosure, the production rate of the photocatalyst may be from about 2,000 TON•h⁻¹ to about 5,000 TON•h⁻¹, but the present disclosure is not limited thereto. For example, the production rate of the photocatalyst maybe about 2,000 TON·h⁻¹ to about 5,000 TON·h⁻¹, about 2,000·TON·h⁻¹ to about 4,500 TON·h⁻¹, about 2,000 TON·h⁻¹ to about 4,000 TON·h⁻¹, about 2,000 TON·h⁻¹ to about 3,500 TON·h⁻¹, about 2,000 TON·h⁻¹ to about 3,000 TON·h⁻¹, about 2,000 TON·h⁻¹ to about 2,500 TON·h⁻¹, about 2,500 TON·h⁻¹ to about 5,000 TON·h⁻¹, about 2,500 TON·h⁻¹ to about 4,500 TON·h⁻¹, about 2,500 TON·h⁻¹ to about 4,000 TON·h⁻¹, about 2,500 TON·h⁻¹ to about 3,500 TON·h⁻¹, about 2,500 TON·h⁻¹ to about 3,000 TON·h⁻¹, about 3,000 TON·h⁻¹ to about 5,000 TON·h⁻¹, about 3,000 TON·h⁻¹ to about 4,500 TON·h⁻¹, about 3,000 TON·h⁻¹ to about 4,000 TON·h⁻¹, about 3,000 TON·h⁻¹ to about 3,500 TON·h⁻¹, or about 3,500 TON·h⁻¹ to about 4,000 TON·h⁻¹, but the present disclosure is not limited thereto. Specifically, the production rate of the photocatalyst may be about 2,000 TON·h⁻¹ to about 4,000 TON·h⁻¹, about 2,000 TON·h⁻¹ to about 3,500 TON·h⁻¹, about 2,000 TON·h⁻¹ to about 3,000 TON·h⁻¹.

In an embodiment of the present disclosure, the selectivity of the photocatalyst may be about 90% or more, but the present disclosure is not limited thereto. For example, the selectivity of the photocatalyst may be about 90% or more, about 92% or more, about 94% or more, about 95% or more, about 96% or more, about 97% or more, about 98% or more, or about 99% or more, but the present disclosure is not limited thereto.

In an embodiment of the present disclosure, the photocatalyst for carbon dioxide reduction may include, but is not limited to, one or more mononuclear transition metal complexes selected from (2-(2-pyridyl)benzothiazole)Ni(pyridine-2-thiolate)₂, (2-(2-pyridyl)benzimidazole)Ni(pyridine-2-thiolate)₂, (2-(2-pyridyl)benzothiazole)Co(pyridine-2-thiolate)₂, (2-(2-pyridyl))benzimidazole)Co(pyridine-2-thiolate)₂, (2-(2-pyridyl)benzothiazole)Mn(pyridine-2-thiolate)₂, (2-(2-pyridyl)benzimidazole)Mn(pyridine-2-thiolate)₂, (2-(2-pyridyl)benzothiazole)Fe(pyridine-2-thiolate)₂, and (2-(2-pyridyl)benzimidazole)Fe(pyridine-2-thiolate)₂. Specifically, in an embodiment of the present disclosure, the photocatalyst for carbon dioxide reduction may be (2-(2-pyridyl)benzothiazole)Ni(pyridine-2-thiolate)₂ or (2-(2-pyridyl)benzimidazole)Ni(pyridine-2-thiolate)₂.

In an embodiment of the present disclosure, the method is performed in a solvent, and the solvent may be a mixed solvent containing water and alcohol, but the present disclosure is not limited thereto. For example, the alcohol may include, but is not limited to, one or more selected from methanol, ethanol, propanol, butanol, pentanol, hexanol, hexanol, octanol, ethanol glycol, and propylene glycol. In an embodiment of the present disclosure, the alcohol may be ethanol.

In an embodiment of the present disclosure, the method may be performed at room temperature, but the present disclosure is not limited thereto.

In an embodiment of the present disclosure, the method may be carried out in a pH range of about 8 to about 14, but the present disclosure is not limited thereto. For example, the pH of the photocatalyst may be about 8 to about 14, about 8 to about 13, about 8 to about 12, about 8 to about 11, about 8 to about 10, about 8 to about 9, about 9 to about 14, about 9 to about 13, about 9 to about 12, about 9 to about 11, about 9 to about 10, about 10 to about 14, about 10 to about 13, about 10 to about 12, about 10 to about 11, about 11 to about 14, about 11 to about 13, about 11 to about 12, about 12 to about 14, about 12 to about 13, or about 13 to about 14, but the present disclosure is not limited thereto. Specifically, the pH of the photocatalyst may be about 8 to about 11, or about 9 to about 11.

### [Best Mode for Invention]

Hereinafter, the present disclosure will be described in more detail with reference to Examples, but the present disclosure is not limited thereto.

### EXAMPLES

<Materials and Measurements>

All reagents were purchased from Aldrich and used without further purification, unless otherwise specified. Water was purified by a Milli-Q purification system. A 2-(2-pyridyl)benzimidazole (pbi) ligand was obtained from Aldrich.

UV-vis spectra were recorded with a Hewlett Packard 8453 spectrophotometer. NMR spectra were recorded at room temperature with a Bruker 300 MHz spectrometer. Hydrogen production was measured by gas chromatography using a DS6200 gas chromatograph (Donam Instrument Inc., Korea) equipped with a Carbosphere 80/100 Mesh, 6 ft×1/8″ OD SS Column (Alltech, Part No.5682PC) and a thermal conductivity detector (TCD).

### Preparation of Complex 1 [2-(2-pyridyl)benzimidazole)Ni(pyridine-2-thiolate)₂]

A solution of 2-(2-pyridyl)benzimidazole (pbi) (0.8 mmol) in 8 mL of acetonitrile was slowly added to a solution containing Ni (NO₃)₂ (H₂O)₆ (0.8 mmol) in acetonitrile (15 mL) over 30 minutes. The color of the solution changed from dark green to light blue. After stirring for 1 hour, a solution containing pyridine-2-thiol (1.6 mmol) and triethylamine (2 mmol) in 10 mL of acetonitrile was slowly added to the solution over 1 hour. The color of the solution changed to yellow-green, and after stirring for an additional hour, a green precipitate was formed. Complex 1 was collected by suction filtration and washed with acetonitrile. The Complex 1 was recrystallized using CH₃CN/ether.

¹H-NMR (300 MHz, DMSO-d₆, ppm):1.1, 6.8, 7.0, 7.2, 7.6, 8, 10.9, 11.4, 14.9, 15.7, 29.7, 31.8, 46.6, 47.5, 29.6, 56.9, 59.6, 67.4, 71.1. ESI-MS: observed at m/z=474.13 for [pbi+2pyS⁻¹+Ni²⁺+H⁺]⁺.

### Preparation of 2-(2-pyridyl)benzothiazole) (pbt)

A ligand, pbt was prepared according to a known procedure. To a solution of pyridine-2-carboxaldehyde (5 mmol) in 20 mL of methanol was added 2-aminothiophenol (5 mmol). The solution was refluxed for 8 hours. Upon cooling the solution to room temperature, a pale yellow solid precipitated. The precipitate was collected by filtration, washed several times with hexane and then with diethyl ether. The solid was recrystallized from hot methanol to obtain pale yellow needles.

### Preparation of Complex 2 [(2-(2-pyridyl)benzothiazole)Ni(pyridine-2-thiolate)₂]

Mononuclear Ni (II) complexes of pyridylbenzothiazole (pbt) were prepared in a manner similar to that described for Complex 1 above.

A solution of pyridylbenzothiazole (pbt) (0.8mmol) in 8 mL of acetonitrile was slowly added to a solution containing Ni (NO₃)₂ (H₂O)₆ (0.8 mmol) in acetonitrile (15 mL) over 30 minutes. The color of the solution changed from dark green to light blue.

After stirring for 1 hour, a solution containing pyridine-2-thiol (1.6 mmol) and triethylamine (2 mmol) in 10 mL of acetonitrile was slowly added to the solution over 1 hour. The color of the solution changed to yellow-green, and after stirring for an additional hour, a green precipitate was formed. The Complex 2 [2-(2-pyridyl)benzothiazole)Ni(pyridine-2-thiolate)₂, (Ni (pbt) (pyS)₂) ] was collected by suction filtration and washed with acetonitrile.

The Complex 2 was recrystallized using CH₃CN/ether and dried in vacuo to give a red orange solid in 65% yield. X-ray quality crystals were grown by diffusing diethyl ether into the acetonitrile solution of the Complex 2 at room temperature.

¹H-NMR (300 MHz, DMSO-d₆, ppm): 1.2, 6.8, 7.6, 8.1, 8.4, 8.8, 10.1, 11.4, 12.6, 16.1, 23.6, 47.1, 48.8, 57.0, 58.2, 61.0, 67.8, 71.6, 73.7. ESI-MS: observed at m/z=490.8 for [pbt+2pyS⁻¹+Ni²⁺+H⁺]⁺.

The Complex 2 [(2-(2-pyridyl)benzothiazole)Ni(pyridine-2-thiolate)₂] exhibited a slightly faster rate and higher yield for formic acid production than the Complex 1 [2-(2-pyridyl)benzimidazole)Ni(pyridine-2-thiolate)₂]

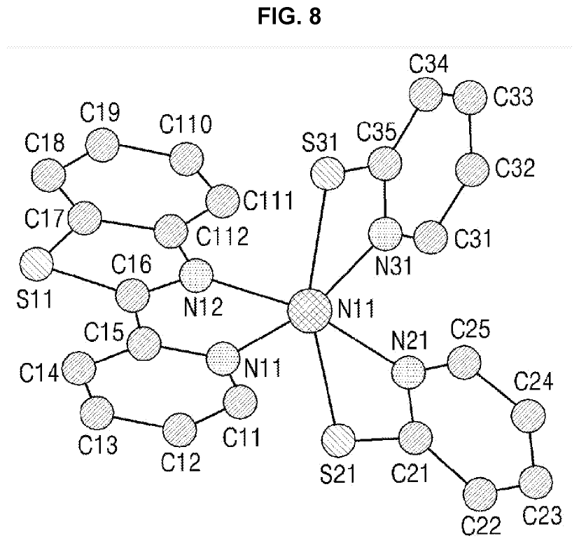

It can be seen that through X-ray analysis of the single crystal of Composite 2, Ni ions are 6-coordinated in a distorted octahedral structure (FIG. 8).

The Complex 1 and the Complex 2 provide the highest TONs (13,100 to 14,000) and high selectivity reported so far for the photoconversion of visible light-driven carbon dioxide into the formic acid.

Table 1 shows the crystal data and structure improvement of Ni (pbt) (pyS)₂ (Composite 2):

TABLE 1

| Chemical formula | C₂₂H₁₆N₄NiS₃ | |
|---|---|---|
| Molecular Weight | 491.28 | |
| Temperature | 296 (2) K | |
| wavelength | 0.71073 Å | |
| Crystal system | Single crystal | |
| Space group | P 1 2₁/nl | |
| Unit cell dimensions | a = 7.5847 (2) Å | α = 90° |
| | b = 25.2162 (6) Å | β = 92.0718 (14)° |
| | c = 10.9933 (3) Å | γ = 90° |

| | |
|---|---|
| Volume | 2101.17 (9) Å³ |
| Z | 4 |
| Density (calculated) | 1.553 g/cm³ |
| Absorption coefficient | 1.239 mm⁻¹ |
| F(000) | 1008 |
| Crystalline nature | Red block |
| Crystal size | 0.120 × 0.220 × 0.200 mm |
| Theta range for data | 1.61° to 28.30° |
| collection | |
| Index range | −10 ≤ h ≤ 10, −33 ≤ K ≤ 33, |
| | −14≤/≤14 |
| Collected reflection | 65446 |
| Independent reflection | 5210 [R (int) = 0.0726] |
| Coverage of Independent | 99.8% |
| reflection | |
| Absorption correction | Multiple Scans |
| Maximum and Minimum | 0.8660 and 0.7900 |
| Transmission | |
| Refinement Method | Full matrix least squares |
| | for F² |
| Data/Restriction/Parameters | 5210/0/271 |
| Fit for F² | 1.051 |
| Final R index [I > 2σ (I)] | R1 = 0.0435, wR2 = 0.0920 |
| R index (all data) | R1 = 0.0682, wR2 = 0.0922 |
| The biggest difference, | 0.829 and −0.283e Å⁻³ |
| Peak and hole | |
| Deviation from R.M.S mean | 0.064 e Å⁻³ |

Table 2 shows the bond length (A) of Ni(pbt) (pyS)₂ (Composite 2):

TABLE 2

| | | | | | |
|---|---|---|---|---|---|
| C11-N11 | 1.323 | (3) | C11-c12 | 1.377 | (4) |
| C12-C13 | 1.376 | (4) | C13-C14 | 1.379 | (4) |
| C14-C15 | 1.384 | (3) | C15-N11 | 1.349 | (3) |
| C15-C16 | 1.468 | (4) | C16-N12 | 1.306 | (3) |
| C16-S11 | 1.732 | (2) | C17-C18 | 1.385 | (4) |
| C17-C112 | 1.402 | (3) | C17-S11 | 1.734 | (3) |
| C18-C19 | 1.368 | (4) | C19-C110 | 1.395 | (4) |
| C21-N21 | 1.350 | (3) | C21-C22 | 1.399 | (4) |
| C21-S21 | 1.731 | (3) | C22-C23 | 1.370 | (4) |
| C23-C24 | 1.379 | (5) | C24-C25 | 1.365 | (4) |
| C25-N21 | 1.336 | (3) | C31-N31 | 1.343 | (3) |
| C31-C32 | 1.367 | (5) | C32-C33 | 1.374 | (5) |
| C33-C34 | 1.367 | (5) | C34-C35 | 1.393 | (4) |
| C35-N31 | 1.348 | (3) | C35-S31 | 1.738 | (3) |
| C110-C111 | 1.369 | (4) | C111-C112 | 1.399 | (4) |
| C112-N12 | 1.392 | (3) | N11-Ni1 | 2.084 | (2) |
| N12-Ni1 | 2.134 | (2) | N21-Ni1 | 2.059 | (2) |
| N31-Ni1 | 2.059 | (2) | Ni1-S21 | 2.4864 | (8) |
| Ni1-S31 | 2.5108 | (8) | | | |

Table 3 shows the bond angle) (°) of Ni (pbt) (pyS)₂ (Composite 2):

TABLE 3

| | | | | | |
|---|---|---|---|---|---|
| N11-C11-C12 | 123.2 | (3) | C13-C12-C11 | 119.2 | (3) |
| C12-C13-C14 | 118.9 | (3) | C13-C14-C15 | 118.5 | (3) |
| N11-C15-C14 | 122.9 | (2) | N11-C15-C16 | 113.3 | (2) |
| C14-C15-C16 | 123.8 | (2) | N12-C16-C16 | 120.7 | (2) |
| N12-C16-S11 | 115.87 | (19) | C15-C16-S11 | 123.41 | (18) |
| C18-C17-C112 | 121.5 | (2) | C18-C17-S11 | 128.8 | (2) |
| C112-C17-S11 | 109.68 | (19) | C19-C18-C7 | 117.9 | (3) |
| C18-C19-C110 | 121.3 | (3) | N21-C21-C22 | 120.3 | (3) |
| N21-C21-S21 | 112.72 | (19) | C22-C21-S21 | 127.0 | (2) |
| C23-C22-C21 | 118.6 | (3) | C22-C23-C24 | 120.6 | (3) |
| C25-C24-C23 | 118.1 | (3) | N21-C25-C24 | 122.7 | (3) |
| N31-C31-C32 | 121.7 | (3) | C31-C32-C33 | 119.0 | (3) |
| C34-C33-C32 | 119.7 | (3) | C33-C34-C35 | 119.5 | (3) |
| N31-C35-C34 | 120.0 | (3) | N31-C35-S31 | 113.3 | (2) |
| C34-C35-S31 | 126.7 | (2) | C111-C110-C19 | 121.3 | (3) |
| C110-C111-Cl12 | 118.3 | (2) | N21-C112-C111 | 126.1 | (2) |
| N12-C112-C17 | 114.3 | (2) | C111-C112-C17 | 119.6 | (2) |
| C11-N11-C15 | 117.3 | (2) | C11-N11-Ni1 | 126.95 | (18) |
| C15-N11-Ni1 | 115.71 | (16) | C16-N12-C112 | 110.9 | (2) |
| C16-N12-Ni1 | 111.55 | (16) | C112-N12-Ni1 | 137.38 | (16) |
| C25-N21-C21 | 119.7 | (2) | C25-N21-Ni1 | 138.1 | (2) |
| C21-N21-Ni1 | 102.07 | (16) | C31-N31-C35 | 119.9 | (2) |
| C31-N31-Ni1 | 137.8 | (2) | C35-N31-Ni1 | 102.30 | (16) |
| N31-Ni1-N21 | 91.37 | (8) | N31-Ni1-N11 | 164.89 | (9) |
| N21-Ni1-N11 | 94.90 | (8) | N31-Ni1-N12 | 97.95 | (8) |
| N21-Ni1-N12 | 166.31 | (8) | N11-Ni1-N12 | 78.55 | (8) |
| N31-Ni1-S21 | 97.96 | (7) | N21-Ni1-S21 | 68.24 | (6) |
| N11-Ni1-S21 | 97.14 | (6) | N12-Ni1-S21 | 100.38 | (6) |
| N31-Ni1-S31 | 68.12 | (6) | N21-Ni1-S31 | 99.10 | (7) |
| N11-Ni1-S31 | 97.27 | (6) | N12-Ni1-S31 | 93.69 | (6) |
| S21-Ni1-S31 | 161.57 | (3) | C16-S11-C17 | 89.28 | (12) |
| C21-S21-Ni1 | 76.87 | (9) | C35-S31-Ni1 | 76.17 | (10) |

<Electrochemistry>

Cyclic voltammetry measurements were performed with a CHI instrument potentiostat/galvanostat (CHI630C) using a one-compartment cell with a glass carbon working electrode, a Pt wire auxiliary electrode, and an Ag/AgCl reference electrode. Non-aqueous electrochemical experiments were performed in 0.1M Bu₄NPF₆ in acetonitrile under an inert atmosphere. Electrochemical experiments performed in 1:1 H₂O: CH₃CN were performed under an inert atmosphere in 0.1 M KNO₃ and compared with aqueous Ag/AgCl.

<Photocatalytic Hydrogen Generation>

The reaction was carried out in a glass cell with a capacity of 26 mL as a reaction vessel containing Complex 1 (or Complex 2, 4 μM), Eosin Y (EY) and TEOA in 2 mL EtOH/H₂O. Prior to irradiation, the solution was purged with argon or carbon dioxide for 5 minutes. A 450 W Xenon Arc (Newport Co.) was used as the light source.

Since other mononuclear Ni complexes containing pyridine-2-thiolate (pyS) were investigated for H₂ photogeneration, the present inventors carried out photolysis of the complex 1/EY/TEOA solution under Ar. Hydrogen production was monitored in real time and quantified by gas chromatography analysis of the headspace gas. As reported for other mononuclear

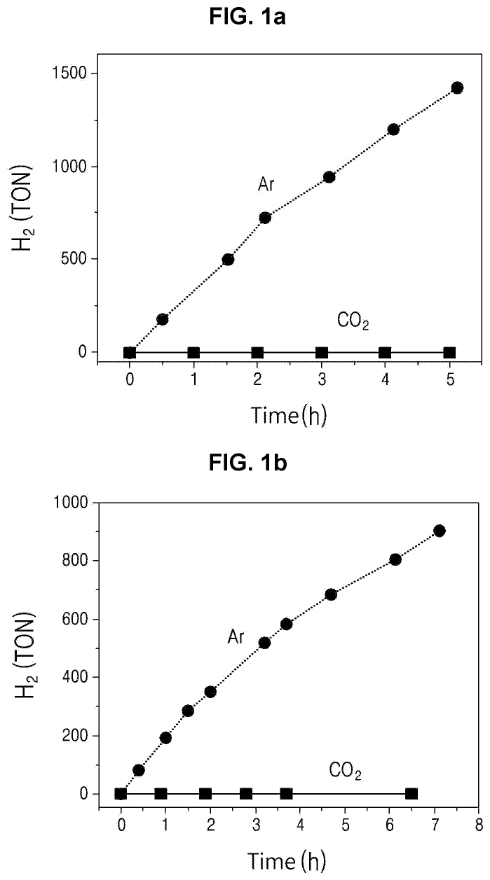

Ni catalysts, a fairly high rate of H₂ production (1,350 TON for 5 hours) was observed at pH 10.7 (FIG. 1a).

FIG. 1a illustrates hydrogen photogeneration using Complex 1, EY (2.0 mM) and TEOA (400 mM) in EtOH:H₂O (1:1, pH=10.7) under argon (●) and carbon dioxide (▪), respectively, and FIG. 1B illustrates hydrogen photogeneration using Complex 2 (4.0 μM), EY (2.0 mM) and TEOA (400 mM) in EtOH:H₂O (1:1, pH =10.7) under argon (●) and carbon dioxide (▪), respectively. Here, the filter is a 420 nm cut-off.

Referring to FIGS. 1a and 1b, the H₂ production was completely quenched when the reaction solution was saturated with carbon dioxide, demonstrating that the carbon dioxide reduction reaction shares the same reactive intermediate in the H₂ generation reaction. That is, the relatively high reactivity of carbon dioxide to Ni (III)-H intermediates takes precedence over proton reduction under the pH conditions investigated In studies involving mononuclear Ni (II) complexes, these Ni (II)-H intermediates have been proposed as reactive species for H₂ generation.

It can be seen that in FIG. 1B, Complex 2 also illustrated similar differences in H₂ production under the same conditions at pH 10.7; but in this case, the H₂ generation reaction in the presence of the Complex 2 is somewhat lower than that in the presence of the Complex 1 of FIG. 1a under Ar (FIG. 1b). The complex Ni (bpy-X₂) (pyS)₂ (X=H, CH₃, OCH₃) photochemically provided 3,100 TON to 7,400 TON of H₂ in EtOH/H₂O for 30 hours.

The photocatalytic reaction of Complex 1 under carbon dioxide was carried out at different pH levels of pH 7.0, 10.7, and 12.7.

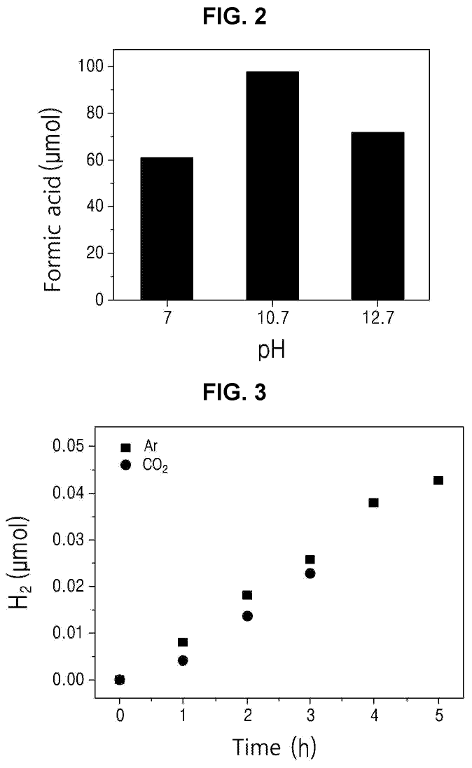

FIG. 2 illustrates photocatalytic carbon dioxide conversion using Complex 1 (4.0 μM), EY (2.0 mM) and TEOA (400 mM) under different pH conditions in EtOH:H₂O (1:1) under carbon dioxide. Referring to FIG. 2, the yield was lower at pH 12.7, as a result of the lower formation rate of Ni—H species due to the lower proton concentration. The pH 7.0 condition also relatively lowered formic acid production. The relatively high proton concentration at pH 7.0 provides better conditions for H₂ production, even in the presence of carbon dioxide. This analysis was confirmed by comparing the H₂ production under conditions under Ar and under carbon dioxide at pH 7.0 (FIG. 3). The formic acid production yield was highest at pH 10.7 (FIG. 2).

FIG. 3 illustrates hydrogen photogeneration using Complex 1 (4.0 μM), EY (2.0 mM) and TEOA (400 mM) in EtOH:H₂O (1:1) at pH 7.0 under argon (▪) and carbon dioxide (●), respectively, using a 420 nm cut-off filter. As illustrated in FIG. 1a, in contrast to the rapid quenching of the H₂ production at pH 10.7, the H₂ production was hardly reduced under carbon dioxide compared to the conditions under Ar.

Other carbon products such as CO were not detected, demonstrating a high selectivity of ˜99% for the formic acid. Control experiments in the absence of Complex 1, EY, or TEOA showed that formic acid was not significantly formed. The maximum TONs in Complex 1 and Complex 2 were limited due to the photobleaching of EY under reaction conditions, as reported in other literatures. In photocatalytic carbon dioxide reduction using Ni complexes of triethylamine and carbine-isoquinoline, only CO, with a high TON of 98,000, was observed. In the most recent studies related to electrocatalytic carbon dioxide conversion with molecular Ni complexes, carbon monoxide has been reported as a major product.

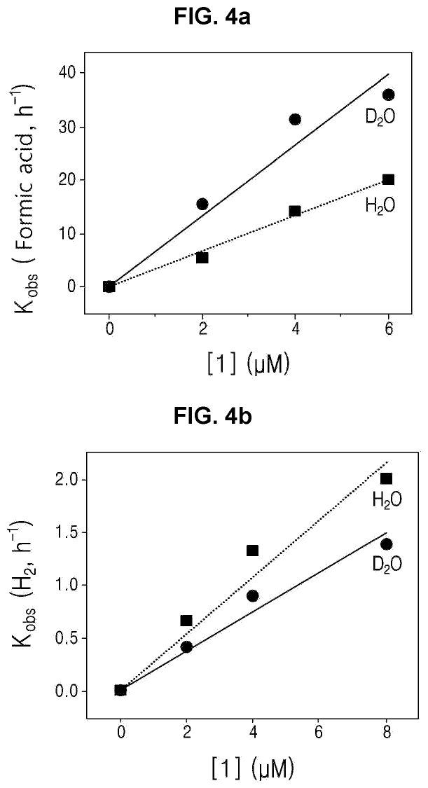

To better understand the properties of the Ni—H intermediates produced during a catalytic cycle, the observed photogeneration rates of formic acid using Complex 1 (or Complex 2) in the presence of EY and TEOA were determined in H₂O/EtOH and D₂O/EtOH saturated with carbon dioxide. When H₂O was replaced with D₂O, the production rate of DCOO⁻ was increased compared to that of formic acid (FIG. 4a).

FIG. 4a illustrates kinetic isotopic effects on formic acid photoproduction by Complex 1, EY (2.0 mM) and TEOA (400mM) in EtOH:H₂O and EtOH/D₂O (1:1, pH=10.7), respectively, under carbon dioxide, and FIG. 4b illustrates kinetic isotopic effects on H₂ photogeneration by Complex 1, EY (2.0 mM) and TEOA (400mM) in EtOH:H₂O and EtOH/D₂O (1:1, pH=10.7)respectively, under Ar. Referring in detail to FIG. 4a, a kinetic study of HCOO⁻/DCOO⁻ production shows substantial inverse kinetic isotopic effects in Complex 1 as k_{H}/k_{D}=0.52 (FIG. 4a). Referring to FIG. 4b, the H₂ production performed using Complex 1 in D₂O under Ar showed general kinetic isotopic effects with a k_{H}/k_{D} ratio of 2.1.

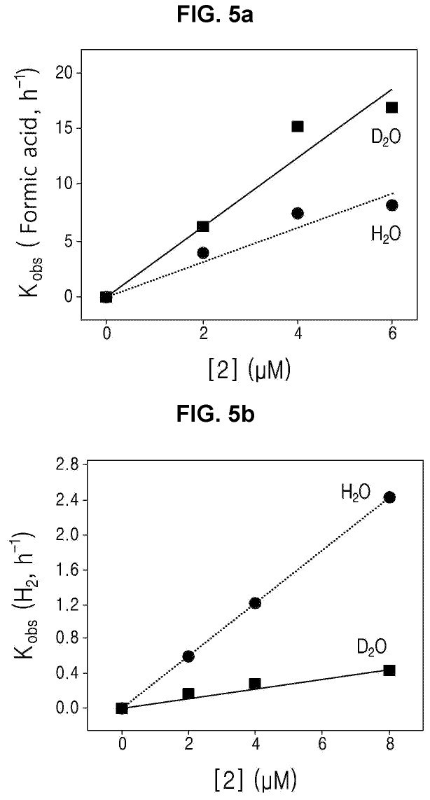

FIG. 5a is a graph illustrating substantial kinetic isotopic effects on formic acid photogeneration by Complex 2, EY (2.0 mM) and TEOA (400 mM) in EtOH:H₂O/D₂O (1:1, pH=10.7) under carbon dioxide, and FIG. 5b is a graph illustrating kinetic isotopic effects on hydrogen photogeneration by Complex 2, EY (2.0mM) and TEOA (400 mM) in EtOH:H₂O/D₂O (1:1, pH=10.7) under Ar. A kinetic study of HCOO⁻/DCOO⁻ production shows substantial inverse kinetic isotopic effects in Complex 2 as k_{H}/k_{D}=0.47 (FIG. 5a).

Similar inverse deuterium isotope effects have been reported for the reaction of carbon dioxide or alkenes with other metal-hydride species. Previous studies have been interpreted based on a rapid attainment of pre-equilibrium according to the rate-limiting hydrogen transition and a zero-point energy difference between the transition state and the basic state of metal hydride. The results of the present disclosure support the production of Ni (II)-H and Ni (II)-D as reactive intermediate species in Complex 1 and Complex 2, and suggest that the formation of Ni—OOCH(D) by the carbon dioxide insertion reaction on Ni—H(D) species is a rate limiting step. However, the H₂ production performed using Complex 1 and Complex 2 in D₂O under Ar showed general kinetic isotopic effects with k_{H}/k_{D} ratios of 2.1 and 4.0, respectively (FIGS. 4b and 5b). Kinetic studies of isolated Ir (III)-H species for H₂ production carried out in H₂O and D₂O likewise showed normal kinetic isotopic effects.

<Photocatalytic Carbon Dioxide Reduction Using Nickel Complexes>

Samples in EtOH/H₂O solution (2 mL) of Complex 1 or Complex 2 (4 μM), EY (2 mM), TEOA (400 mM) were placed in a 26 mL glass cell. The solution was bubbled with carbon dioxide prior to irradiation. Formic acid was monitored by HPLC (YL 9100) on a column (Inertsil ODS-3V, 5 μm 4.6×150 mm) using a H₂PO₄ solution (0.15%) as eluent and a UV detector (λ=210 nm).

First, the quantum efficiency (QE) of the overall catalytic photoredox cycle for carbon dioxide reduction is determined using the following eqauation:

$$AQY ( \% ) = \frac{HCOOH molecules ⨯ 2}{incident photons} ⨯ 100$$

For quantum efficiency measurements, an Xe lamp (450 W) with a 420 nm cut-off filter was used. Here, the number of incident photons may be calculated from an incident photon flux of 1.45×10²¹ photons cm²h⁻¹ and an irradiation area of 0.0004 m². The incident light intensity was determined using a Newport 842-PE actinometer. After 8 hours of photolysis, 1.06×10⁻⁸ moles of HCOOH were produced. The calculated quantum yields of a visible light molecular photosensitizer system are 1.02% (Ni (pbt)) and 0.87% (Ni (pbi)), respectively.

AQY (%) based on 1 hour, pbi=4.36%, pbt=2.53%.

The apparent quantum yield (AQY) for CO/H₂ generation was measured using the same photochemical experimental setup. The intensity of light irradiation was measured to be 2.6 mWcm⁻² (CEAulight, AULTTP4000), and the irradiated area was 1.0 cm².

A photocatalyst for carbon dioxide reduction including a mononuclear transition metal complex may optionally provide formic acid with high efficiency [14,000 turnover number], and optionally provide a high catalytic selectivity of ˜99%. In addition, the photocatalyst for carbon dioxide reduction including the mononuclear transition metal complex maybe used to completely inhibit undesirable proton reduction pathways in photocatalytic reactions with mononuclear transition metals under carbon dioxide, and design a fuel production process through sunlight for artificial photosynthesis.

A series of photocatalytic experiments using the Complex 1 and the Complex 2 were carried out using EY as a photosensitizer and TEOA as a sacrificial electron donor in EtOH/H₂O (1:1) saturated with carbon dioxide under irradiation with visible light (420 nm cut-off filter) at room temperature. The Complex 1 provided formic acid as a major carbon product, and the formic acid was quantitatively analyzed at the end of photolysis by high performance liquid chromatography. The formic acid production rates obtained by Complex 1 and Complex 2 were 3,000 TON·h⁻¹ and 2,500 TON·h⁻¹, respectively, which are the highest turnover frequencies (TOFs) for formic acid production reported so far for photocatalytic carbon dioxide reduction by first-row transition metal molecular catalysts.

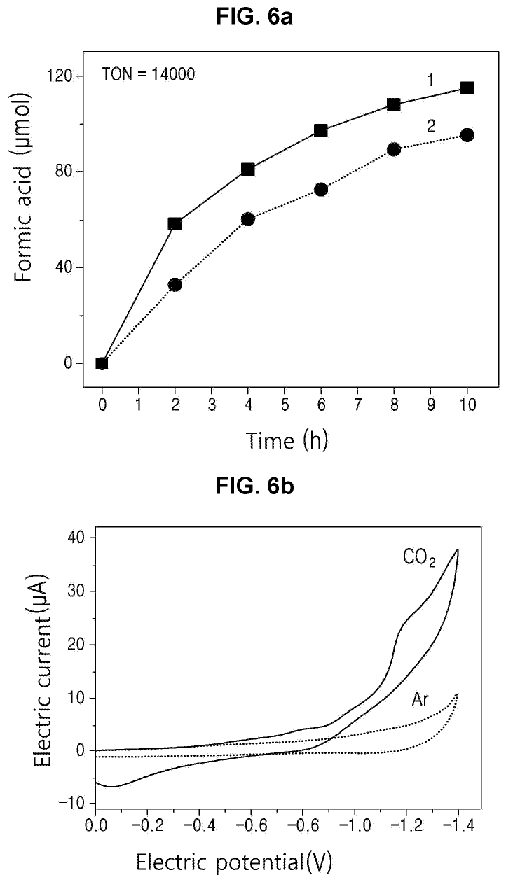

FIG. 6a illustrates photocatalytic reduction of carbon dioxide to formic acid using Complex 1 (▪) and Complex 2 (●) (4.0 μM) in the presence of EY (2.0 mM) and TEOA (400 mM) in EtOH/H₂O (1:1, pH=10.7) at room temperature, and FIG. 6b is a cyclic voltammetry curve of Complex 1 under Ar (dotted line) and carbon dioxide (solid line) in 0.1 M KNO₃ (aq) (GC electrode, 100 mVs⁻¹).

For FIG. 6a, the maximum TONs observed for the catalysts were 14,000 and 13,100, respectively, upon 8 hours of irradiation for Complex 1 and Complex 2, respectively. The quantum yield in Complex 1 was measured to be 4.8% at 420 nm based on two photons and formic acid generated for 1 hour per molecule of carbon dioxide. In Composite 1 and Composite 2, these TONs are the best reported so far for photocatalytic reactions using molecular Ni catalysts and other first-row transition metal complexes.

Referring to FIG. 6b, the cyclic voltammetry curve of Complex 1 in the aqueous KNO3 solution was obtained under Ar (dotted line) and carbon dioxide (solid line). The Ni^{II/I} redox couple of Composite 1 showed about −1.2 V under Ar; but a high catalytic current was observed for the Complex 1 solution saturated with carbon dioxide (FIG. 6b).

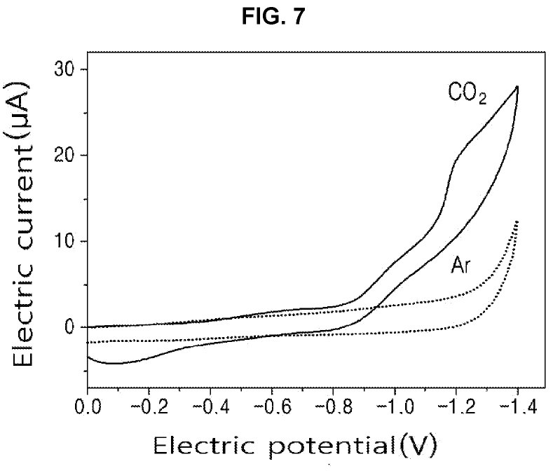

FIG. 7 illustrates a cyclic voltammetry curve (GC electrode, 100 mV/s) of Complex 2 in 0.1 M KNO₃ (aq) under argon (dotted line) and CO₂ (solid line). As can be seen from FIG. 7, Complex 2 showed similar results to Complex 1 of FIG. 6b; but its catalytic current was somewhat lower than that of Composite 1 under the same conditions (FIG. 7). Their catalytic current is related to the carbon dioxide reduction, which is consistent with the photogeneration result of formic acid. The enhanced catalytic current at the Ni (II) reduction potential demonstrates that the Complex 1 and the Complex 2 are the cause of carbon dioxide reduction.

The present inventors also investigated the quenching rate of EY release by the Complex 1. The fluorescence intensity of the EY at 630 nm was plotted for various concentrations of Complex 1 to obtain a quenching rate constant of 1.7×10⁹ s⁻¹. The excited state of EY was also quenched by TEOA, which represents reductive quenching, and oxidative quenching of EY also occurred by the Complex 2.

The initial photochemical step is reductive quenching, wherein the excited state of EY was reduced by reaction with TEOA. Although this path predominates because the relative concentration of TEOA is 10⁵ times higher than that of the Ni catalyst; oxidative quenching is possible because the concentration of EY is 500 times higher than that of the Ni catalyst. The quenching results indicate that oxidative and reductive quenching occurs for electron transfer between the Ni composite, EY, and TEOA.

Based on all the observations mentioned above, the present inventors proposed the following mechanism. The following mechanisms represent the proposed mechanisms of H₂ photogeneration and carbon dioxide photoconversion into formic acid by Ni (II) catalysts, EY, and TEOA.

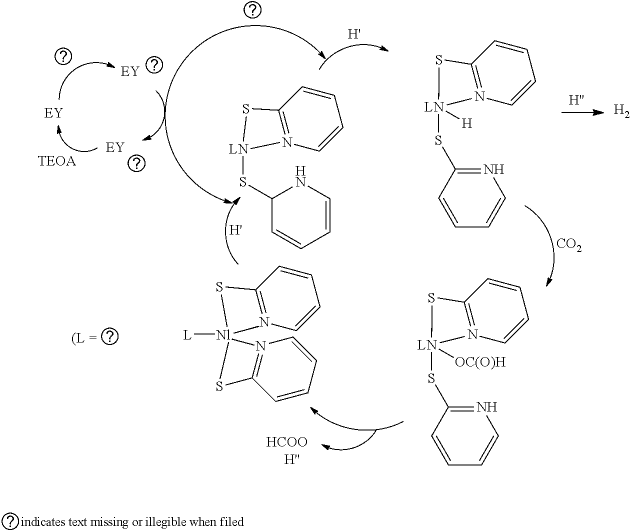

The photoexcited EY transferred two electrons successively to the Ni center to generate Ni (II)-hydride species. The photocatalytic mechanism for carbon dioxide reduction to the Ni center begins with protonation of pyridyl nitrogen, which may occur with de-chelation. Ni-H species may participate in one of two reduction reactions depending on the reaction conditions. The protonated pyridyl group continuously transfers H⁺ to the proposed Ni (II)-H intermediate, which is formed in the cycle for carbon dioxide reduction and H₂ generation, as suggested in the H₂ generation process with other Ni complexes of pyS. In the present of carbon dioxide at a high pH, the Ni (II)-H intermediate has a higher tendency to undergo a rate-limiting insertion reactions with carbon dioxide, thereby generating Ni (II)-formic acid species and then releasing formic acid. This carbon dioxide insertion reaction with molecular metal complexes of Co and Ni has been previously proposed for formic acid formation. In the absence of carbon dioxide, the Ni−H species participate in the reduction of protons to produce H₂.

¹H-NMR spectra of Complex 1 and Complex 2 show proton resonances (5) ranging from 0 ppm to 80 ppm, as reported for other 6-coordinated mononuclear Ni (II) complexes. Complex 1and Complex 2 were also characterized by electrospray ionization mass spectrometry (ESI-MS), providing [Ni²⁺+pbi+2pyS⁻+H⁺]⁺ at m/z=474.13 and [Ni²⁺+pbt+2pyS⁻+H⁺]⁺ at m/z=490.80, respectively.

The following structures shows the chemical structures of the Complex 1 and the Complex 2:

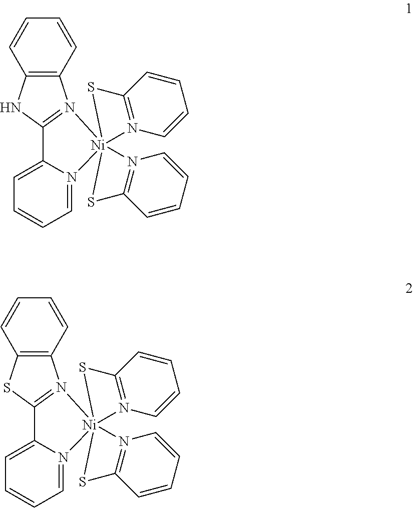

FIG. 8 illustrates an ORTEP photograph of Composite 2. When reviewing the structure of the Composite 1 and the Composite 2, the bonding distances of Ni—N and Ni—S and the trans conformation of the two sulfurs of the two pyS ligands are similar to those reported for related Ni complexes generated from pyS and bpy derivatives. Referring to FIG. 8, in the case of pbt of the Complex 2, the imine nitrogen (N12) was coordinated to the Ni center instead of S11. Due to the asymmetric structure and size of benzothiazole related to pyridine, the bonding length of Ni—N12 is longer than that of Ni—N11. The other Ni—N bonding distances of Composite 2 are similar to those reported for other related Ni composites. For Complex 1, the imine nitrogen of pbi may coordinate to the Ni (II) center instead of the secondary amine, as reported for other pbi complexes of Ni (II), Mn (II), and Cu (II) and supported from ESI-MS of Complex 1.

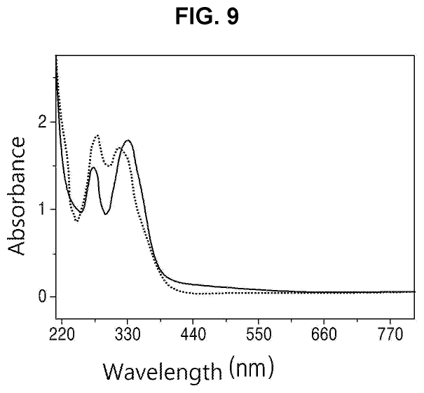

FIG. 9 illustrates absorption spectra of 0.1 mM Complex 1 (solid line) and Complex 2 (dotted line) in H₂O/EtOH. Referring to FIG. 9, it can be seen that the spectrum of Complex 1 (solid line) shows high energy absorption bands at 274 nm (ε=14,960) and 332 nm (ε=17,990) ; the spectrum of Complex 2 (dotted line) shows bands at 278 nm (ε=18,690) and 316 nm (ε=17,230), which correspond to spin-tolerant intraligand (p-p*) transitions (FIG. 9).

The present inventors have demonstrated that novel molecular Ni (II) complexes may causephotocatalytic conversion of carbon dioxide into formic acid. Two Ni complexes of 2-(2-pyridyl)benzimidazole and 2-(2-pyridyl)benzothiazole were active for H₂ photogeneration under Ar but showed selective formic acid production under carbon dioxide in an aqueous/organic solvent mixture. Formic acid production occurred with high selectivity compared to proton reduction under high pH conditions. A Ni-pbi composite was used to obtain a high TOF of 3,000 h⁻¹ and a high TON of 14,000, and the selectivity to formic acid was >99%. These results demonstrate a remarkable photocatalytic system for high and selective conversion of carbon dioxide using earth-abundant metal composites, organic photosensitizers and sacrificial electron donors under visible light energy irradiation.

The photocatalytic reaction mechanism for carbon dioxide reduction at the Ni center begins with the protonation of the pyridyl nitrogen, which may occur with de-chelation. The proposed Ni (II)-H intermediate and proton reduction, which is directly related to carbon dioxide, is supported by a protonated pyridyl group carrying H. The rate-limiting step has been proposed in which Ni—OC(O)H is formed by carbon dioxide insertion into the Ni—H intermediate, which was supported by reverse kinetic isotope effects. The highest activity was obtained with the Ni-pbi complex, which exhibited the highest TON reported for molecular photochemical systems without noble metals. The present disclosure provides an adjustable route for formic acid production with excellent energy efficiency using earth-abundant elements in the process of converting environmental pollutants into more useful organic compounds, as the Ni (II) catalyst also exhibits substantially improved carbon dioxide photoconversion compared to H₂ generation.

The description of the present disclosure stated above is for illustration, and it will be understood by those of ordinary skill in the art to which the present disclosure pertains that the present disclosure may be easily modified into other specific forms without changing the technical spirit or essential features of the present disclosure. Therefore, it is to be understood that the embodiments described above are illustrative rather than being restrictive in all aspects. For example, each component described as a single type may be implemented in a dispersed form, and likewise components described as distributed may also be implemented in a combined form.

It should be interpreted that the scope of the present disclosure will be indicated by the claims rather than the above-mentioned description and the meaning and scope of the claims, and all changes or modifications derived from their equivalents are included in the scope of the present disclosure.

1. A mononuclear transition metal complex represented by the following Formula 1:
L¹-M-(L²)₂ [Formula 1]
wherein
M is a mononuclear transition metal of Ni, Fe, Mn, or Co,
L¹ is 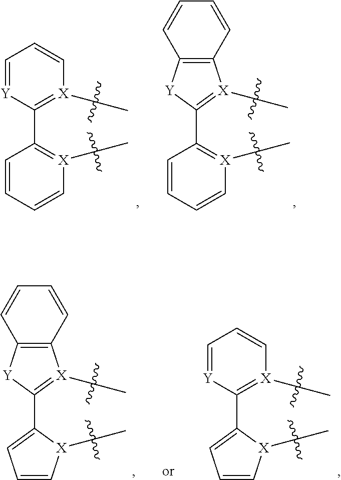
L² is 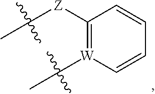
in L¹,
X is N or P,
Y is —CH, —N—, —NH, —S—, or —O—;
in L²,
Z is —O—, —S—, or—NH,
W is —P— or —N—;
the aryl group and/or heteroaryl group included in L¹ or L² is substituted or unsubstituted, and when the aryl group and/or the heteroaryl group is substituted, the substituent is one or more selected from a linear or branched C₁-C₆ alkyl group, a C₃-C₆ cycloalkyl group, a C₂-C₆ heterocycloalkyl group, a linear or branched C₁-C₆ alkoxy group, a halogen group, an amine group, or a linear or branched C₁-C₆ alkylamine group, and
the broken line means that the ligand is coordinated to the mononuclear transition metal.

2. The mononuclear transition metal complex of claim 1, wherein L¹ is 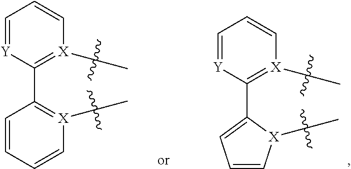
and Y is —CH or —N—.

3. The mononuclear transition metal complex of claim 1, wherein L¹ is 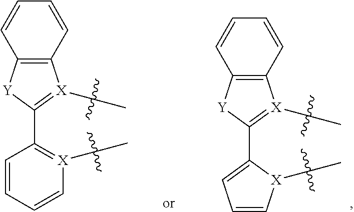
and Y is NH, —S—, or —O—.

4. The mononuclear transition metal complex of claim 1, wherein the mononuclear transition metal complex represented by Formula 1 is (2-(2-pyridyl)benzothiazole)Ni(pyridine-2-thiolate)₂, (2-(2-pyridyl)benzimidazole)Ni(pyridine-2-thiolate)₂, (2-(2-pyridyl)benzothiazole)Co(pyridine-2-thiolate)₂, (2-(2-pyridyl))benzimidazole)Co(pyridine-2-thiolate)₂, (2-(2-pyridyl)benzothiazole)Mn(pyridine-2-thiolate)₂, (2-(2-pyridyl)benzimidazole)Mn(pyridine-2-thiolate)₂, (2-(2-pyridyl)benzothiazole)Fe(pyridine-2-thiolate)2, or(2-(2-pyridyl)benzimidazole)Fe(pyridine-2-thiolate)2.

5. The mononuclear transition metal complex of claim 1, wherein the transition metal of the mononuclear transition metal complex is 6-coordinated in a distorted octahedral structure.

6. A photocatalyst for carbon dioxide reduction, comprising a mononuclear transition metal complex represented by the following Formula 1:
L¹-M-(L²)₂ [Formula 1]
wherein
M is a mononuclear transition metal of Ni, Fe, Mn, or Co,
L¹ is 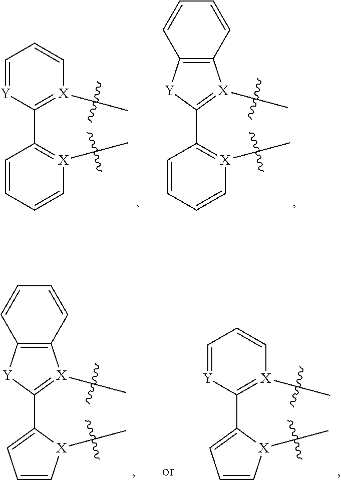
L² is 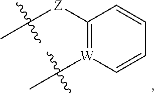
in L¹,
X is N or P,
Y is —CH, —N—, —NH, —S—, or —O—;
in L²,
Z is —O—, —S—, or—NH,
W is —P— or —N—;
the aryl group and/or heteroaryl group included in L¹ or L² is substituted or unsubstituted, and when the aryl group and/or the heteroaryl group is substituted, the substituent is one or more selected from a linear or branched C₁-C₆ alkyl group, a C₃-C₆ cycloalkyl group, a C₂-C₆ heterocycloalkyl group, a linear or branched C₁-C₆ alkoxy group, a halogen group, an amine group, or a linear or branched C₁-C₆ alkylamine group, and
the broken line means that the ligand is coordinated to the mononuclear transition metal.

7. The photocatalyst for carbon dioxide reduction of claim 6, wherein the photocatalyst for carbon dioxide reduction is to reduce carbon dioxide to formic acid.

8. The photocatalyst for carbon dioxide reduction of claim 6, further comprising a cocatalyst, wherein the cocatalyst includes one or more selected from eosin Y, Ru(bpy)₃, C₃N₄, CdS, CdSe, and triethanolamine.

9. The photocatalyst for carbon dioxide reduction of claim 6, wherein L¹ is 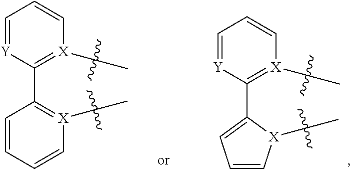
and Y is —CH or —N—.

10. The photocatalyst for carbon dioxide reduction of claim 6, wherein L¹ is 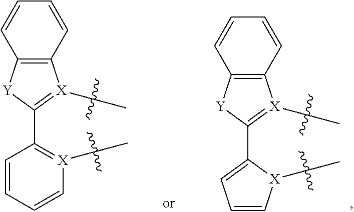
and Y is NH, —S—, or —O—.

11. The photocatalyst for carbon dioxide reduction of claim 6, wherein the mononuclear transition metal complex is one or more selected from (2-(2-pyridyl)benzothiazole)Ni(pyridine-2-thiolate)₂, (2-(2-pyridyl)benzimidazole)Ni(pyridine-2-thiolate)₂, (2-(2-pyridyl)benzothiazole)Co(pyridine-2-thiolate)₂, (2-(2-pyridyl))benzimidazole)Co(pyridine-2-thiolate)₂, (2-(2-pyridyl)benzothiazole)Mn(pyridine-2-thiolate)₂, (2-(2-pyridyl)benzimidazole)Mn(pyridine-2-thiolate)₂, (2-(2-pyridyl)benzothiazole)Fe(pyridine-2-thiolate)₂, and(2-(2-pyridyl)benzimidazole)Fe(pyridine-2-thiolate)₂.

12. A method for reducing carbon dioxide to formic acid, the method comprising using the photocatalyst for carbon dioxide reduction of claim 6.

13. The method of claim 12, wherein a production rate of the photocatalyst is from about 2,000 TON·h⁻¹ to 5,000 TON·h⁻¹.

14. The method of claim 12, wherein a selectivity of the photocatalyst is 90% or more.

15. The method of claim 12, wherein the method is carried out at a pH ranging from 8 to 14.

16. The method of claim 12, wherein the photocatalyst for carbon dioxide reduction includes one or more mononuclear transition metal complexes selected from (2-(2-pyridyl)benzothiazole)Ni(pyridine-2-thiolate)₂, (2-(2-pyridyl)benzimidazole)Ni(pyridine-2-thiolate)₂, (2-(2-pyridyl)benzothiazole)Co(pyridine-2-thiolate)₂, (2-(2-pyridyl))benzimidazole)Co(pyridine-2-thiolate)₂, (2-(2-pyridyl)benzothiazole)Mn(pyridine-2-thiolate)₂, (2-(2-pyridyl)benzimidazole)Mn(pyridine-2-thiolate)₂, (2-(2-pyridyl)benzothiazole)Fe(pyridine-2-thiolate)₂, and(2-(2-pyridyl)benzimidazole)Fe(pyridine-2-thiolate)₂.

17. The method of claim 12, wherein the method is carried out in a solvent, and
the solvent is a mixed solvent containing water and alcohol.

18. The method of claim 12, wherein the method is carried out at room temperature.
